(19) United States
(12) Patent Application Publication
(10) Pub. No.: US 2013/0078661 A1
(43) Pub. Date: Mar. 28, 2013

(54) Compounds and Methods for FRET Based Measurement of Enzyme Activity

(71) Applicants: Sriram Neelamegham, Getzville, NY (US); Kannayakanahalli Dayananda, Amherst, NY (US)

(72) Inventors: Sriram Neelamegham, Getzville, NY (US); Kannayakanahalli Dayananda, Amherst, NY (US)

(73) Assignee: The Research Foundation for The State University of New York, Amherst, NY (US)

(21) Appl. No.: 13/701,930

(22) PCT Filed: Jun. 6, 2011
(86) PCT No.: PCT/US2011/039232

§ 371 Date: Dec. 4, 2012

Related U.S. Application Data
(60) Provisional application No. 61/351,318, filed on Jun. 4, 2010.

(51) Int. Cl. C07K 14/755; C12Q 1/37

(52) U.S. Cl. CPC C07K 14/755; C12Q 1/37
USPC 435/24; 530/383

(57) ABSTRACT

Provided are compositions and methods for Fluorescence/Forster Resonance Energy Transfer (FRET) analysis of cleavage of Von Willebrand Factor (VWF) polypeptides by the metalloprotease ADAMTS13. The polypeptides contain: i) a first fluorescent protein ii) a VWF amino acid sequence and iii) a second fluorescent protein. The VWF amino acid can contain any of a variety of amino acid substitutions. The method involves contacting a sample that contains ADAMTS13 and using FRET based measurements to determine ADAMTS13 cleavage of the recombinant polypeptide.

### CROSS-REFERENCE TO RELATED APPLICATIONS

This application claims priority to U.S. provisional patent application No. 61/351,318, filed on Jun. 4, 2010, the disclosure of which is incorporated herein by reference.

### GOVERNMENT FUNDING

This invention was made with government support under HL76211 and HL76258 awarded by the National Institutes of Health. The government has certain rights in the invention.

### FIELD OF THE INVENTION

The present invention relates generally to substrates for measuring enzyme activity and more specifically to novel substrates for Fluorescence/Forster Resonance Energy Transfer (FRET) analysis of cleavage of Von Willebrand Factor (VWF) by the metalloprotease ADAMTS13.

### BACKGROUND OF THE INVENTION

VWF is a large, multimeric glycoprotein found in human circulation. The hemostatic function of this protein increases with its molecular mass. The size of VWF is regulated by the constitutively active blood metalloprotease ADAMTS13 (a disintegrin and metalloprotease with thrombospondin type 1 motif 13). This enzyme cleaves the Tyr¹⁶⁰⁵-Met¹⁶⁰⁶ bond, which is constitutively buried within the A2-domain of VWF in its wild type state.

Inherited/familial or acquired defects in ADAMTS13 function result in the inefficient cleavage of VWF and the presence of high molecular mass VWF in circulation. The presence of high molecular mass VWF contributes to the spontaneous binding of platelet receptor GpIbα to VWF via the VWF-A1 domain, platelet activation, aggregation and microthrombi formation. Such microthrombi can cause vessel occlusion, ischemia and organ failure leading to a condition called thrombotic thrombocytopenic purpura (TTP). Accurate and rapid strategies to quantify ADAMTS13 activity could thus serve as a prognostic marker of TTP. However, currently available approaches for determining ADAMTS13 activity (Tripodi, et al. J Thromb Haemost 6 (2008) 1534-41) suffer from a variety of drawbacks.

For example, Kokame et al. (British Journal of Haematology, (2005) Vol. 129, pp 93-100) described the failed attempts of several groups to provide suitable compositions and methods for measuring the VWF-cleavage activity of ADAMTS13. Kokame et al. attempted to provide an improved method of measuring ADAMTS13 activity which employed a fluorgenic substrate (VWF73, containing the 73-amino acids from D1596 to R1668 of VWF) for use in FRET analysis. However, this substrate must be chemically synthesized so that the Q1599 residue at the P7 position can be converted to a 2,3-diaminopropionic residue (A2pr) that is modified with a 2-(N-methylamino)benzoyl group (Nma). It also requires that the N1610 residue at the P5′ position be converted to A2pr modified with a 2,4-dinitrophenyl group (Dnp). This configuration provides for excitation of the Nma group at 340 nm, which permits transfer of fluorescence resonance energy to the Dnp quencher. If ADAMTS13-mediated cleavage between Y1605 and M1606 occurs, the energy transfer quenching of the fluorescence does not occur, which results in emission of fluorescence at 440 nm from Nma. This methodology is currently in widespread use for measuring ADAMTS13 activity in human blood or plasma samples, but at least in part because of the single fluorophore/quencher configuration, the assays lacks linearity, requires an internal control, and is a cumbersome substrate to produce and use. More recently, Zhang et al. (Analytical Biochemistry (Notes & Tips); (2006) Vol. 358, pp 298-300) described synthesis of a recombinant 73-amino acid VWF peptide for use in FRET-based analysis of ADAMTS13 activity. However, this requires engineering two cysteines into the VWF sequence so that they replace P1611 and Q1599. The recombinant protein is produced in cell culture and purified, after which the engineered cysteines are reduced and modified with identical fluorescein moieties. Thus, the method of Zhang et al. requires introduction of cysteines for the purpose of providing fluorescein docking sites, followed by production, purification and extensive manipulation of the protein so that the fluorescein moiety can be attached in two places. This is an inefficient process and moreover results in yet another assay which requires an internal control, since use of the identical fluorescein moiety on each recombinant cysteine necessitates interpretation of self-quenching effects. Accordingly, there is an ongoing need for improved methods and substrates for use in determining ADAMTS13 cleavage of VWF. The present invention meets these and other needs.

### SUMMARY OF THE INVENTION

The present invention provides compositions and methods for FRET analysis of VWF polypeptides by ADAMTS13. The polypeptides comprise in sequence i) a first fluorescent protein ii) a VWF amino acid sequence which comprises at least amino acids 1596-1668, inclusive, of SEQ ID NO:1 and, iii) a second fluorescent protein. In various embodiments, the recombinant polypeptide is not longer than 229 amino acids and is at least at least 73 amino acids in length.

Each polypeptide described herein can contain any conservative amino acid substitution, insertion or deletion that would be expected to not adversely affect ADAMTS13 cleavage. In addition to such changes, in various embodiments the polypeptides of the invention can comprise at least one mutation selected from the group of mutations in Table 1. In an embodiment of the invention, the at least one mutation is a change in Pro 1645 to any amino acid that is not Pro.

The recombinant polypeptides can be made by any suitable techniques and reagents. In various embodiments, prokaryotic (i.e., E. coli) or eukaryotic (i.e., mammalian or insect cells) expression systems which produce the polypeptides from expression vectors can be used. The invention accordingly includes all polynucleotide sequences encoding the polypeptides, including but not necessarily limited to such polynucleotides in expression vectors.

The invention also provides compositions comprising a recombinant polypeptide of the invention, and optionally including an ADAMTS13 protease. In one embodiment, such a composition further comprises a denaturant at a particular concentration. For example, the denaturant can be included in any amount from 0.1M to 8M, inclusive and including all integers to the tenth decimal place there between. The compositions may further comprise a composition that is a biological sample or a composition derived from a biological sample. In certain embodiments, the biological sample comprises bodily fluids, such as isolated blood or isolated plasma.

A method that is useful for determining ADAMTS13 protease activity is also provided. The method involves contacting a recombinant polypeptide of the invention with a composition comprising ADAMTS13 protease and analyzing ADAMTS13 cleavage of the recombinant polypeptide by FRET analysis. The FRET analysis takes into account fluorescence from the first and second fluorescent proteins. A change in ratio of the fluorescence from the first and the second fluorescent proteins over time is indicative of ADAMTS13 protease activity. In one embodiment, a FRET ratio from the first and the second fluorescent proteins that is lower relative to a reference FRET ratio is indicative that the individual from whom the biological sample was obtained has or is at risk for a disease that is positively correlated with abnormally high molecular weight VWF. In one embodiment, the disease is TTP.

### DESCRIPTION OF THE FIGURES

FIG. 1. VWF-A2 FRET proteins. A. Five FRET proteins were expressed and purified from E. coli. In these, Venus (Ven) and Cerulean (Cer) flank truncated fragments of the VWF-A2 domain. A His-tag is present for protein purification. The size of the A2-insertion increases from 77 amino acids for XS-VWF (eXtra-Small-VWF) to 175 for L-VWF (Large-VWF). XS-VWF(AA) is identical to XS-VWF except that the Y¹⁶⁰⁵-M¹⁶⁰⁶ amino acids in the ADAMTS13 cleavage site are replaced by A¹⁶⁰⁵-A¹⁶⁰⁶. B. Relative positions of Cerulean (cyan) and Venus (yellow) in the family of FRET proteins are shown annotated in the VWF-A2 crystal (3 GBX). Distances between Cerulean and Venus insertion sites in the crystal structure are listed. C. Silver stain showing the purity of VWF-A2 FRET proteins.

FIG. 2. Fluorescence spectra and ADAMTS13 cleavage. 104 of each of the VWF-A2 FRET proteins was incubated for 1 h at 37° C. either in the presence or absence of concentrated recombinant ADAMTS13. No denaturant was added. A-E. Samples diluted 60-fold were analyzed using the fluorescence spectrophotometer using 435 nm excitation wavelength. All spectra were normalized by arbitrarily setting YFP fluorescence intensity to 10,000 (excitation=485 nm; emission=530 nm). F. Selected samples at 1 h were subjected to western blot analysis, using anti-his pAb for detection. XS-VWF but not XS-VWF(AA) was cleaved by recombinant ADAMTS13. Data are representative of >3 experiments.

FIG. 3. Enzyme kinetics. Various concentrations of XS-VWF and S-VWF were incubated with 20% plasma from normal human donors for up to 2 h. FRET ratio measured at various times was used to generate Lineweaver-Burk plot.

FIG. 4. Detection of plasma ADAMTS13 levels by XS-VWF. Various dilutions of human plasma were incubated with 1.5 μM XS-VWF or FRETS-VWF73 under conditions optimized for the latter reagent. A. FRET ratio varied linearly with plasma ADAMTS13 activity for XS-VWF. B. FRETS-VWF73 fluorescence readout varied non-linearly with plasma ADAMTS13 activity. Data are representative of >3 experiments.

FIG. 5. Measurement of low ADAMTS-13 activity. Plasma was obtained from normal human blood (100% ADAMTS13 activity). A portion of this was heat-inactivated to obtain 0% ADAMTS13 activity plasma. Normal plasma and heat-inactivated plasma were then mixed to obtain samples with a variety of ADAMTS13 activities. For example, 40% ADAMTS13 activity plasma was obtained by mixing normal plasma and heat-inactivated plasma in 4:6 proportion. Following this, 204 of the plasma samples with a variety of enzyme activities were mixed with 2 μM XS-VWF in 100 μL reaction volume. XS-VWF cleavage was measured at various times. 10% ADAMTS13 activity was detected at 30 min.

FIG. 6. Effect of urea. A. 2 μM each of the VWF-A2 FRET proteins was incubated with varying urea concentrations in the absence of ADAMTS13. FRET ratio, measured at 1 h, increased with urea concentration. B. Concentrated recombinant ADAMTS13 was added to 1 μM VWF-FRET proteins in the presence of varying urea concentrations for 2.5 h. Samples were then diluted in 20-fold excess buffer for additional 5 min prior to FRET measurement. XS-VWF and S-VWF were cleaved efficiently in the absence of urea. Cleavage rate decreased at urea>2.4M. The extent of L- and M-VWF cleavage increased with urea concentration though neither substrate was fully cleaved in the available time. *p<0.05 with respect to sample without ADAMTS13. ^{†}p<0.05 with respect to both samples that lack urea. C. Western blot analysis of 3 h samples performed using the protocol in FIG. 2F, are consistent with fluorescence data.

FIG. 7. Pre-denaturation with urea. 10 μM L-VWF or 40 μg/mL multimeric plasma VWF was incubated either in the presence or absence of 4M urea at 37° C. for 1 h. Following this, VWF samples were diluted 10-fold into cleavage buffer containing concentrated ADAMTS13 and varying urea amounts for 24 h. A. FRET ratio was measured similar to FIG. 4B. Pre-denaturation of L-VWF with 4M urea enhanced proteolysis rates. B. Western blot of L-VWF samples pre-denatured with urea prior to ADAMTS13 addition show >90% proteolysis. C and D. Western blot of plasma VWF that was either not subjected to (panel C) or was subjected to urea pre-denaturation (panel D). Pre-denaturation is required for the efficient cleavage of both L-VWF and plasma VWF.

FIG. 8. Analysis of mutant VWF-A2 FRET proteins. A. Schematic of representative mutant VWF-A2 FRET proteins. B. Cleavage of mutant FRET protein by recombinant ADAMTS13 in the presence of 1M urea (Western blot results). C. Cleavage of substrates upon addition of 20% human plasma for 2 hours in the presence of 2M urea (error bars are standard deviations for multiple donors).

### DESCRIPTION OF THE INVENTION

The present invention provides compositions and methods for measuring ADAMTS13 protease activity. The compositions comprise a recombinant polypeptide which serves as a substrate for FRET-based measurement of ADAMTS13 protease cleavage.

The polypeptides of the invention comprise sequentially i) a first fluorescent protein ii) a VWF amino acid sequence which comprises at least VWF amino acids 1596-1668 of SEQ ID NO:1; and, iii) a second fluorescent protein. The VWF amino acid can comprise the wild-type human sequence, or the wild-type human sequence with one or more amino acid substitutions, insertions or deletions. Each VWF polypeptide described herein can comprise one or more amino acid substitutions, such as those set forth in Table 1, and is still considered to be a “VWF polypeptide.” Further, each VWF polypeptide described herein can comprise any conservative amino acid substitution, such as any conservative amino acid substitution which would not be expected to interfere with DAMTS13-mediated cleavage of the polypeptide. Polypeptides comprising amino acid substitutions in the human VWF sequence can provide for improved sensitivity of ADAMTS13 activity relative to wild type VWF when used in the method of the invention.

Nucleic acids encoding each polypeptide disclosed herein, as well as expression vectors comprising such nucleic acids are encompassed within the invention.

The method of the invention comprises contacting a recombinant polypeptide of the invention with a sample comprising ADAMTS13 and determining ADAMTS13-mediated cleavage of the recombinant polypeptide. A change in FRET ratio over time is indicative of ADAMTS13-mediated cleavage. If the ADAMTS13 that is being analyzed is present in or is derived from a biological sample and the FRET ratio differs from a reference (normal) FRET ratio such that the ADAMTS13-mediated cleavage is less efficient than the reference, it is indicative that the individual from whom the sample was obtained or derived has or is at risk for a disorder that is positively correlated with abnormally high molecular weight VWF. Such disorders include but are not necessarily limited to TTP. The invention also provides for studying the effects of various amino acid modifications on VWF structure by analyzing ADAMTS13 cleavage of VWF-derived polypeptides comprising various amino acid changes relative to the wild-type sequence.

The sequence of normal human VWF is provided in SEQ ID NO:1. VWF amino acids positions are assigned according to convention that is well recognized in the art, with amino acid 1 denoting the start of the VWF signal peptide. Since ADAMTS13 is known to cleave VWF between the Tyr1605-Met1606 bond, “Y1605” and “M1606” designate the positions of the Tyr and Met between which ADAMTS13 cleaves. The conventional nomenclature for positions of other VWF amino acid positions can be readily ascertained by, for example, their relative locations to the cleavage site. For each amino acid in each VWF polypeptide sequence (whether modified or not) described herein, those skilled in the art will readily be able to determine the position of the VWF amino acid according to the conventional nomenclature.

In one embodiment, the polypeptides of the invention comprise or consist of the human VWF amino acid sequence from 1596-1668 of SEQ ID NO:1. The human VWF sequence consisting of VWF 1596-1668 is:

(SEQ ID NO: 2)
DREQAPNLVYMVTGNPASDEIKRLPGDIQVVPIGVGPNANVQELERIG
WPNAPILIQDFETLPREAPDLVLQR.

In one embodiment, the polypeptides of the invention comprise or consist of the human VWF amino acid sequence from amino acids 1594-1670. The human VWF sequence consisting of VWF 1594-1670 is: QGDREQAPNLVYMVTGNPASDEIKRLPGDIQVVPIGVGPNANVQELERIGWPNAPILIQ DFETLPREAPDLVLQRCC (SEQ ID NO:3). The polypeptide consisting of VWF amino acids 1594-1670 (SEQ ID NO:3) is referred to herein as “XS-VWF”.

In various embodiments, the polypeptides of the invention can comprise up to 229 amino acids of VWF, and may or may not comprise amino acid substitutions. In one embodiment, a polypeptide of the invention does not comprise more than 229 amino acids of VWF (with or without amino acid changes). In one embodiment, a polypeptide of the invention comprises or consists of SEQ ID NO:4, which represents the amino acid sequence of human VWF from 1458 to 1686 and is as follows:

(SEQ ID NO: 4)
CDLAPEAPPPTLPPHMAQVTVGPGLLGVSTLGPKRNSMVLDVAFVLEG
SDKIGEADFNRSKEFMEEVIQRMDVGQDSIHVTVLQYSYMVTVEYPFS
EAQSKGDILQRVREIRYQGGNRTNTGLALRYLSDHSFLVSQGDREQAP
NLVYMVTGNPASDEIKRLPGDIQVVPIGVGPNANVQELERIGWPNAPI
LIQDFETLPREAPDLVLQRCCSGEGLQIPTLSPAPDC.

The polypeptides of the invention can be fragments of SEQ ID NO:4. The fragments can be a minimum of 73 amino acids in length, and can include all sizes of polypeptides that are fragments of SEQ ID NO:4 that are between 73 and 228 amino acids, inclusive, and including all integers there between. Each VWF polypeptide described herein that is suitable for use in determining ADAMTS13 activity contains VWF Tyr 1605 and Met 1606 Met.

In certain embodiments, the polypeptides of the invention that are fragments of SEQ ID NO:4 can comprise VWF amino acids 1596-1668, and thus these polypeptides are at least 73 amino acids in length. Such fragments of SEQ ID NO:4 can comprise VWF amino acids 1594-1670, and thus these polypeptides are at least 77 amino acids in length. In other embodiments, the polypeptides consist of or comprise VWF amino acids 1496-1670 (“L-VWF”), 1530-1670 (“M-VWF”), or 1558-1670 (“S-VWF”). As described above, each VWF polypeptide described herein may comprise one or more amino acid substitutions that are set forth in Table 1.

TABLE 1

| | Metal | | Vicinal | |
|---|---|---|---|---|
| Type 2A | binding | Glycosylation | Cysteines | Cis-Proline |
| A1500E | D1596A | E1515 | R1668 | P1645A |
| L1503Q,P | R1597W | G1574 | C1669G | P1645G |
| G1505E, R | D1498A | | C1670G | |
| S1506L | A1600X | | CC1669-70GG | |
| F1514C | N1602A | | | |
| K1518E | | | | |
| V1539E | | | | |
| L1540P | | | | |
| S1543F | | | | |
| Q1556R | | | | |
| L1562P | | | | |
| L1580P | | | | |
| G1579R | | | | |
| R1583W | | | | |
| R1597 | | | | |
| G, W, Q, L | | | | |
| V1604F | | | | |
| V1607D | | | | |
| G1609R | | | | |
| S1613P | | | | |
| D1614G | | | | |
| P1627H | | | | |
| I1628T | | | | |
| G1629R | | | | |
| V1630E | | | | |
| E1638K | | | | |
| L1639P | | | | |
| P1648S, R | | | | |

Those skilled in the art will recognize that, generally, amino acid changes that affect glycosylation will not affect peptides produced from prokaryotic expression systems but can nevertheless be included in polypeptides made by them. In preferred embodiments, the glycosylation mutations are included in polypeptides made by expression in vitro in mammalian systems.

In various embodiments, the polypeptides of the invention can comprise only one, or only a specific combination of mutations set forth in Table 1. All combinations of mutations specified in Table 1 are included within the scope of the invention.

In certain embodiments, the polypeptides of the invention comprise a change in P1645. The change in P1645 can be to any amino acid that is not Pro. For example, any naturally occurring non-Pro amino acid can be substituted in the VWF P1645 position.

In specific embodiments, P1645 is changed to an Ala or Gly. The polypeptides can comprise as the only change in sequence a change of P1645, or additional changes at other positions can be included.

With respect to the amino acid substitutions set forth in Table 1, the mutations shown in the Type 2A column are generally based on VWD type 2A disease. For example, several mutations in the A2 domain of VWF result in a condition called Von Willebrand disorder (VWD) type 2A, which is a common bleeding disorder. VWD Type 2A mutations can cause structural changes in the A2 domain by changing the H-bond network, calcium binding sites, or by exposing hydrophobic regions that are buried inside the A2 domain. Such changes can result in instability of the A2 domain and expose the cryptic ADAMTS13 cleavage site (Tyr1605-Met1606). Thus, including one or more of the Type 2A disease-related mutations is expected to enhance ADAMTS13 cleavage efficiency.

With respect to substitutions proximal to the α6 chain of VWF-A2 (Vicinal Cysteines and Cis-Pro columns of Table 1), the VWF-A2 domain also has vicinal cysteines at position 1669 and 1670 that form a intra-molecular disulfide link. This linkage results in a rigid octo-shape ring structure. In addition, VWF-A2 has an unusual cis-Pro1645. As described in the Examples presented herein, mutations to the vicinal cys (CC1669-70GG) and cis-Pro (P1645A) residues result in enhanced cleavage via ADAMTS-13.

With respect to mutations based on calcium binding sites and N-linked glycosylation (the Metal binding and Glycosylation columns of Table 1), there are two N-linked glycosylation sites in the A2 domain. Mutation of N1574 resulted in the efficient cleavage of VWF by ADAMTS13 (McKinnon, T. A. et al. (2008) Blood, Vol. 111, 3042-3049) and there may be a VWF-A2 calcium binding site (Zhou, M., et al. (2011 Blood, published online Mar. 8, 2011). Therefore, it is expected that mutations in these sites will enhance cleavage of the polypeptides described herein.

In demonstrating the present invention, we incorporated in the A2 domain of VWF variants of the cyan (CFP) and yellow fluorescent protein (YFP) that are called Cerulean and Venus respectively. Although 77-175 amino acids of the VWF A2 domain separate Cerulean and Venus in the primary sequence of these molecules, the insertion sites of these green fluorescent protein (GFP) variants lie spatially within 2.5 nm in VWF-A2 crystal structure. Due to this design, the novel family of polypeptides presented here for the first time exhibit FRET properties. Together, these polypeptides are termed “VWF-A2 FRET” proteins. The smallest FRET molecule we generated (“XS-VWF”) detects ADAMTS13 in blood within 5-10 min in the absence of denaturant (K_{M}=4 μM), and it can readily detect plasma protease levels down to <10% of normal levels. This sensitivity rivals or exceeds the resolution of FRETS-VWF73 (described in Kokame et al.), another fluorescence based biosensor of ADAMTS13 activity. Thus, in one embodiment, the method of the invention can be performed without including denaturant in the ADAMTS13 assay. In other embodiments, the method of the invention can be performed using a denaturant concentration greater than 0 M but less than 4M, including all real numbers there between, such as all integers to the tenth decimal place. In certain embodiments, the denaturant is at a concentration greater than 0 M but less than 2.4M, including all real numbers there between, such as all integers to the tenth decimal place. In one embodiment, the denaturant is urea is substituted by chaotropic agents, which include but are not necessarily Guanidinium Chloride and Lithium Perchlorate. In alternative or additional aspects, the temperature or pH can be altered relative to conventional ADAMTS13 assay, and/or the polypeptide substrate can be subjected to any other reagent that disrupt the hydrogen or salt-bridge network in the A2-domain of VWF and that results in exposure of the cryptic 1605Tyr-1606Met scissile bond. In this regard, we determined that VWF-A2 conformation changed progressively, and not abruptly, upon increasing urea concentration. While proteins with 77 and 113 VWF-A2 residues were cleaved in the absence of denaturant, 4M urea was desirable for the efficient cleavage of larger constructs. The invention accordingly provides compositions comprising polypeptides of the invention and a denaturant at any denaturant concentration disclosed herein for performing the method of the invention.

Other advantages of the VWF-A2 FRET proteins compared to FRETS-VWF73 include but are not necessarily limited to the following: i) The VWF-A2 FRET proteins can be produced in large amounts by E. coli in soluble form, unlike FRETS-VWF73 that requires chemical synthesis. We also efficiently synthesize the proteins in mammalian systems, which provides for appropriate glycosylation. Synthesis of the previously reported FRETS-VWF73 is complicated since the synthetic peptide is long; ii) VWF-A2 FRET proteins contain two fluorophores (Venus and Cerulean) unlike FRETS-VWF73 which has one fluorophore and a quencher. Thus, data generated by the polypeptides described herein can be presented in the form of “FRET ratio” (as discussed further below). While the absolute value of this parameter can vary between instruments since detector sensitivity settings at individual wavelengths can be tuned by users, the FRET ratio of the ADAMTS13 cleaved substrate provided by the invention to that of the original intact protein will be approximately 2.75±0.15, regardless of the measuring device and instrument settings. Since FRETS-VWF73 is based on the principles of quenching (whether a distinct quencher or identical self-quenching fluorophores are used), cleavage measurements vary with instrument settings and assay conditions. Importantly, it is not straightforward to translate the measured fluorescence change to estimate % substrate cleavage. However, the instant polypeptides permit excitation at 435 nm (which produces lower plasma auto-fluorescence) and emissions at 485 and 530 nm. In contrast, VWF73 relies on excitation at 340-350 nm (which produces high auto-fluorescence) and emissions at 440-450 nm. The use of higher wavelengths may also partially improve substrate performance in plasma samples having high levels of bilirubin since this yellow product quenches light with peak absorbance at 450 nm. iii) Due to the large separation between fluorophores in VWF-A2 FRET, unlike FRETS-VWF73 where the fluorophore and quencher are separated by ten amino acids, the instant polypeptides can be applied to study changes in VWF-A2 structure under a variety of conditions in addition to proteolysis. iv) It is noteworthy that it would not be possible to insert fluorescent proteins in the positions used for chemical modification of VWF73 with fluorophores/quenchers as disclosed in Kokame et al. (modifying Q1599 and N1610) or Zhang et al. (modifying P1611 and Q1599) and then use these substrates for ADAMST13 activity assays since it is well known in the art that the remaining VWF sequence would be too short to be recognized by ADAMST13, despite the presence of the VWF Tyr 1605 and Met 1606 Met in such substrates. In this regard, the Examples presented herein demonstrate that introduction of Venus/Cerulean itself did not alter the ability of VWF-A2 to undergo ADAMTS13 mediated cleavage. The smallest FRET protein, XS-VWF, detected plasma ADAMTS13 activity down to 10% of normal levels. Tests of acquired and inherited TTP were able to be completed within 30 minutes. Therefore, the present invention provides a method for determining ADAMTS13 activity that is up to 90% lower than the enzyme activity observed in a pool of normal human plasma (i.e., plasma with no apparent coagulation defect). “Normal” ADAMTS13 activity can be determined by measuring ADAMTS13 activity from individuals who do not have a disorder that is positively correlated with defects in VWF, or from a reference, such as a standardized curve, protease titrations, or other normalization of enzyme activity that will be known to the skilled artisan.

Overall, it will be apparent to those skilled in the art that our VWF-A2 FRET proteins can be applied both for the rapid diagnosis of plasma ADAMTS13 activity, and as a tool to study VWF-A2 conformation dynamics using the novel approach of having two distinct fluorescent protein amino acid sequences flanking the VWF polypeptide sequences.

To perform the method of the invention, a recombinant polypeptide described herein is contacted with a sample comprising ADAMTS13 protease. Cleavage results in an increase in physical distance between the first and second fluorescent proteins, which accordingly alters the FRET signal from each fluorescent protein. Thus, in one embodiment, the activity of the ADAMTS13 protease is determined by measuring initial and subsequent changes in emission profiles from the first and second fluorescent proteins. Excitation of the first and second fluorescent proteins can be measured separately or in combination. Emission patterns from the first and second fluorescent proteins can be analyzed to determine a FRET ratio. The FRET ratio changes over time during cleavage of substrate that is contacted and cleaved by the ADAMTS13 protease.

In one embodiment, when the fluorescent proteins are Cerulean and Venus, for example, the ratio of emitted light intensity at 485 nm (primarily by Cerulean) versus 540 nm (primarily by Venus), when the polypeptide is excited at 420 nm yields a FRET ratio. The FRET ratio is inversely related to FRET efficiency because the ratio increases upon proteolysis. Those skilled in the art will recognize, given the benefit of the present disclosure, that other fluorescent proteins can be similarly utilized and concomitant FRET ratios calculated and interpreted in a similar manner.

With respect to the fluorescent proteins, it is expected that any fluorescent proteins can be used. In certain embodiments, the fluorescent proteins are Cyan fluorescent protein (CFP) and Yellow fluorescent protein (YFP), which are considered to be derivatives of Green Fluorescent Protein (GFP).

In specific embodiments the fluorescent proteins are Cerulean and Venus, which have amino acid and polynucleotide coding sequences that are well known in the art and can be produced as components of fusion proteins using conventional techniques. In these proteins, blue/cyan fluorescence protein (BFP/CFP) or its variants (such as Cerulean) may be retained and YFP can be replaced with RFP variants such as dsRed, mRFP1, tagRFP, mCherry, mOrange, HcRed, Tomato. Alternatively YFP or its variants (such as Venus) can be retained and CFP can be changed to RFP or its variants, such as dsRed, mRFP1, tagRFP, mCherry, mOrange, HcRed, or Tomato. CFP or YFP can also be changed to GFP or variants of GFP. For example, polynucleotide sequences encoding the fluorescent proteins can be designed to be encoded in the same translational reading frame with any other amino acid sequence as desired and expressed as a fusion protein using standard reagents and methods. Either fluorescent protein can be included at the N or C terminus of the VWF polypeptides of the invention. Further, either or both of the fluorescent protein amino acid sequences can be flanked at one or both ends by other amino acids. In one embodiment, a fluorescent protein amino acid sequence is contiguous with an amino acid sequence that is used to facilitate isolation and/or purification of the fusion protein. For example, one of the fluorescent protein amino acid sequences can be immediately adjacent to a HIS tag, or could be separated from a HIS tag by spacer or other amino acid sequence(s). Alternatively or in addition to a HIS tag, a FLAG sequence can be included in the polypeptides of the invention. Alternative strategies include the use of IgG (immunoglobulin), MBP (Maltose binding proteins) or GST (Glutathione S-transferase) tagged proteins.

The VWF polypeptides comprising the first and second florescent proteins (fusion proteins) of the invention can be made using techniques well known to those skilled in the art. For example, any DNA sequence encoding any fusion protein of the invention can be made using standard techniques and inserted into any of a number of expression vectors. Suitable expression vectors have been described in the literature and many are commercially available. Likewise, a wide variety of expression systems are known in the art and are commercially available, including prokaryotic and eukaryotic systems. Thus, in non-limiting examples, bacterial, fungal, insect and mammalian expression systems can each be used to make the fusion proteins of the invention. In one embodiment, the expression system uses E. colii. In another embodiment, the expression system uses HEK cells, other mammalian or insect cells. The fusion proteins can be isolated from the expression system using conventional techniques and can be purified to any desired degree of purity.

The invention also provides polynucleotide sequences encoding each and every protein described herein. The polynucleotide sequence encoding the fusion proteins may be present as described above in an expression vector, or in any other type of vector, such as a shuttle vector, cloning and sub-cloning vectors, and the like. The polynucleotides may be provided in a composition comprising a cell culture. Thus, the invention provides cell cultures comprising polynucleotides encoding the fusion proteins of the invention. The polynucleotides may also be provided with other reagents useful for expressing and/or purifying the proteins from the cell culture or for using the proteins encoded by the polynucleotides.

The fusion proteins can be used in the method of the invention in association with any composition of matter, device or system that is suitable for determining ADAMTS13 activity. In one embodiment, the fusion protein is provided in physical association with a solid matrix. The solid matrix may be present in a multi-well assay plate, beads, or any other form or format that is suitable for keeping the fusion protein in a position whereby ADAMTS13 activity can be determined.

The method of the invention is suitable for performing on a biological sample obtained from an individual of any age or gender. The biological sample can be any biological sample that would be expected to contain ADAMTS13. In one embodiment, the sample is blood. In another embodiment, the sample is plasma. The method may be performed once, or a series of tests may be performed to, for example, monitor an individual's response to a treatment.

In one embodiment, the biological sample is obtained from an individual and used directly in an ADAMTS13 activity assay of the invention. In another embodiment, the biological sample is obtained from the individual and subjected to a processing step before the biological sample and/or ADAMTS13 separated from the biological sample is used in determining the ADAMTS13 activity. In some embodiments, the processing step can be carried out to isolate, concentrate, and/or purify the ADAMTS13 for analysis.

In one embodiment, the invention comprises fixing the result of performing the method of the invention in a tangible medium of expression, such as a digitized computer record. The invention further comprises communication of the result of the performing the method of the invention to a health care provider. The invention also comprises receiving the result of the assay and making a treatment recommendation for the individual based at least in part on the result.

The invention also provides kits comprising a fusion protein of the invention. The kits may comprise reagents suitable for use in FRET assays. The kits may further comprise components for biological sample collection and instructions for performing the method of the invention.

The invention is further illustrated by the following Examples and Figures, which are intended to exemplify but not limit the invention.

### Example 1

This Example provides a description of the materials and methods used to obtain the results presented in the Examples that follow it.

VWF-A2 FRET Constructs:

Fusion proteins containing the A2 domain of VWF were expressed in E. coli. Many of the proteins were fused to variants of either YFP (Venus) or CFP (Cerulean). These fluorophores are monomeric since they incorporate the A206K mutation, and they exhibit higher brightness, lower photobleaching, higher extinction coefficient and quantum yield when compared to the original CFP and YFP (M. A. Rizzo, et al. Nat Biotechnol 22 (2004) 445-9; T. Nagai, K. et al Nat Biotechnol 20 (2002)) 87-90). PCR primers used for cloning steps are listed in Table 2.

TABLE 2

| Name of the primer | Primer sequence (5′-3′) * | Use |
|---|---|---|
| FP1_AgeI sense | GGGCGGACCGGTATGGTGAGCAAGGGCGAGGAGCTG | Citrine into pCSCG-A2-FH |
| | (SEQ ID NO: 5) | |
| FP1_AgeI antisense | CGCGCCACCGGTCTTGTACAGCTCGTCCATGCCGAG | |
| | (SEQ ID NO: 6) | |
| His7STOPHind3_Rev | CGCGCGCAAGCTTAATGGTGATGGTGATGGTGATG | Citrine-A2 from pCS-CG into |
| | GCC (SEQ ID NO: 7) | pRSETB |
| NdeI_Citrine_For | GCGGCGGCATATGGTGAGCAAGGGCGAGGAGCTG | Citrine-A2 from pCS-CG into |
| | (SEQ ID NO: 8) | pRSETB, Venus into pRSET-Citrine-A2-FH |
| FP2_AgeI antisense | CGCGCACCGGTCTCGTCCATGCCGAGAGTGATCCC | Venus into pRSET-Citrine-A2-FH |
| | (SEQ ID NO: 9) | |
| FP1_HpaI sense | [Phos]AACATGGTGAGCAAGGGCGAGGAGCTG | Cerulean into pRSET-Ven-A2-FH |
| | (SEQ ID NO: 10) | |
| FP1_BstBI antisense | CCGCCGTTCGAACTTGTACAGCTCGTCCATGCCG | |
| | (SEQ ID NO: 11) | |
| AgeIVWF1594For | GTGTGTACCGGTCAGGGTGATCGGGAGCAGGCGCC | A2 (XS-VWF) forward primer |
| | (SEQ ID NO: 12) | |
| AgeIVWF1558For | CGTAGTACCGGTAAAGGGGACATCCTGCAGCGGG | A2 (S-VWF) forward primer |
| | (SEQ ID NO: 13) | |
| AgeIVWF1530For | CGCGCGACCGGTGTGGGCCAGGACAGCATCCACG | A2 (M-VWF) forward primer |
| | (SEQ ID NO: 14) | |
| AgeIVWF1496For | GGGATTACCGGTGTTCTGGATGTGGCGTTCGTC | A2 (L-VWF) forward primer |
| | (SEQ ID NO: 15) | |
| HpaIVWF1670Rev | [Phos]AACGCAGCACCTCTGCAGCACCAGG | Reverse primer for all of the above |
| | (SEQ ID NO: 16) | A2-FRET conststructs |
| VWFNdeI1481F | GCGCGCGCATATGGGGCTCTTGGGGGTTTCGACC | For making pRSET-A2-Cer-H |
| | CTG (SEQ ID NO: 17) | |
| VWFHpaI1668R | [Phos]AACCCTCTGCAGCACCAGGTCAGGAGC | |
| | (SEQ ID NO: 18) | |
| VWF_YM1605_6AA_rev | GATTTCCCGTGACAGCAGCGACCAGGTTG | ¹⁶⁰⁵Y-¹⁶⁰⁶M to ¹⁶⁰⁵A-¹⁶⁰⁶A |
| | (SEQ ID NO: 19) | mutation primer |

*Restriction enzyme sites: GTTAAC: HpaI; [Phos]ACC: HpaI compatible end; ACCGGT: AgeI; CATATG: NdeI; AAGCTT: HindIII; TTCGAA: BstBI

The plasmids pCS-CG (plasmid number 12154), pVenus-GalT (11931), pCerulean-VSVG (11913) and pRSET FLII12Pglu-600u (13563) were purchased from Addgene (Cambridge, Mass.). Initially, pCS-CG was modified by replacing the green fluorescence protein (GFP) sequence that it originally contained with the Kozak sequence followed by the VWF signal peptide, AgeI and HpaI restriction enzyme sites, the FLAG epitope, TEV cleavage site, poly 6×-histidine tag and a stop codon. A BstBI enzyme site is present between FLAG and TEV. This vector where the CMV promoter and VWF signal peptide drive protein expression is designated pCSCG-KZK-SS-FLAG-His. Using full length VWF cDNA in pcDNA3.1 (Invitrogen) as a template, the A2-domain of VWF (amino acids 1481-1668) was cloned into pCSCG-KZK-SS-FLAG-His using the AgeI and HpaI sites for insertion. The resulting plasmid is called pCSCG-A2-FH. Next, the KpnI-HindIII fragment containing Citrine from pRSET FLII12Pglu-600u was cloned into pUC19. Citrine was amplified in this vector with primers flanked by AgeI, and it was inserted in the correct orientation at the N-terminus of VWF-A2 in pCSCG-A2-FH. The resulting vector is called pCSCG-Citrine-A2-FH, and it was used for mammalian protein expression.

For bacterial expression, the region containing Citrine, VWF-A2, FLAG, TEV site, and poly-histidine from pCSCG-Citrine-A2-FH was amplified by PCR using primers with NdeI and HindIII overhangs, and this was cloned into pRSET-B (Invitrogen). During PCR, sequence encoding for an extra histidine was introduced and thus all bacterial proteins have a his₍₇₎ tag. Venus (Ven) from pVenus-GalT with primers containing NdeI and AgeI overhangs also replaced Citrine in pRSET-Citrine-A2-FH to produce pRSET-Ven-A2-FH. Next, Cerulean (Cer) was amplified from pCerulean-VSVG using primers with phosphorylated HpaI compatible overhangs and BstBI, and this was fused at the C-terminus of the A2 domain in pRSET-Ven-A2-FH. Insertion of Cerulean resulted in loss of the FLAG epitope. This last vector is designated pRSET-Ven-A2-Cer-H. Based on the VWF-A2 crystal structure (3GXB), regions pertaining to 1496-1670 (L-VWF), 1530-1670 (M-VWF), 1558-1670 (S-VWF) and 1594-1670 (XS-VWF) were amplified by PCR with forward primers containing AgeI and phosphorylated reverse primers containing HpaI compatible restriction site overhangs. These PCR products that encode for truncated fragments of the full A2-domain were individually cloned into pRSET-Ven-A2-Cer-H. This resulted in a series of products where truncated forms of A2 were flanked by Venus and Cerulean. A2-domain containing Cerulean alone was also created by PCR amplifying A2 (1481-1668) with primers containing NdeI and HpaI compatible restriction site overhangs and replacing Venus and L-VWF in the vector pRSET-Ven-(L-VWF)-Cer-H. This product is called pRSET-A2-Cer-H. To generate XS-VWF(AA), we mutated the ¹⁶⁰Tyr(Y)-¹⁶⁰⁶Met (M) sequence in XS-VWF (1594-1670) to ¹⁶⁰⁵Ala (A)-¹⁶⁰⁶Ala (A). To this end, a mega primer was first generated using a reverse primer containing the YM to AA mutation and a forward primer encoding for the start of the XS-VWF sequence. The PCR product was purified using Qiaquick PCR kit (Qiagen, Valencia, Calif.), and this was used along with a reverse primer encoding for the 3′ section of XS-VWF during the second PCR step. The final PCR product was cloned into pRSET-Ven-(L-VWF)-Cer-H to replace L-VWF. DNA sequencing was performed to verify all plasmid constructs described above.

Expression and Purification of VWF-A2 FRET Proteins from E. coli:

pRSET vectors encoding for VWF-A2 constructs were transformed into E. coli BL21 Star™ or other strains. Single colonies were scaled up to 1 L in LB broth supplemented with 50 μg/mL ampicillin. Cells were grown to OD₆₀₀=0.6 at 37° C., and then induced with 1 mM IPTG for 12 h at 30° C. Following this, all protein purification steps described below were performed at 4° C. First, the cells from 1 L culture were centrifuged at 2000 g for 20 min, washed with phosphate buffered saline, and then resuspended in 10 mL of 20 mM HEPES buffer (pH-7.4) containing 300 mM NaCl, 10 mM imidazole and EDTA-free protease inhibitor cocktail (Roche Diagnostics, Indianapolis, Ind.). Cells were then lysed by 3 cycles of freeze-thaw. Lysozyme (10 μg/mL) was added for additional 30 min to further facilitate lysis. Following this, the genomic DNA was sheared using a Branson sonifier (Danbury, Conn.). Lysate was separated from the cell debris by centrifuging at 20,000 g for 30 min. The pellet was discarded and the supernatant was filtered through a 0.22 μM syringe filter. The lysate was then passed through HisTrap HP column (GE Healthcare, Piscataway, N.J.) that was equilibrated with HEPES buffer used for the cell lysis. The column was washed with 5-10 volumes of 20 mM HEPES buffer (pH-7.4) containing 300 mM NaCl and 68 mM imidazole. Protein was eluted with 20 mM HEPES (pH-7.4) containing 300 mM NaCl and 300 mM imidazole.

Plasma VWF and ADAMTS13:

Multimeric human VWF was purified from plasma cryoprecipitate using known techniques (H. Shankaran, et al. Blood 101 (2003) 2637-45). Recombinant human ADAMTS13 and diluted human plasma were used as a source of ADAMTS13 activity. Human plasma was obtained from blood drawn from healthy non-smoking volunteers into sodium citrate. All human subject protocols were approved by the University at Buffalo Institutional Review Board. Platelet poor plasma (PPP) was obtained using standard methods (H. Shankaran, et al. Blood 101 (2003) 2637-45). These samples were stored at −80° C. and thawed at 37° C. prior to use.

Proteolysis Assays:

Typically, FRET assays were performed in 404 volume containing 1 μM VWF-A2 FRET protein and 84 concentrated recombinant ADAMTS13 diluted in cleavage buffer (50 mM Tris pH 8.0, 12.5 mM CaCl₂). Urea was added to cleavage buffer in some cases. In other instances, protein was denatured with 4M urea for 1 h at 37° C. prior to addition of ADAMTS13. Comparison of FRETS-VWF73 (Peptides International, Louisville, Ky.) and XS-VWF was performed in a 100 μL cleavage reaction mixture containing 1.5 μM of substrate and 64, of plasma.

At fixed times, in some cases, VWF-A2 FRET fluorescence spectra was obtained using a spectrophotometer (model F-2500, Hitachi, Tokyo, Japan) with excitation wavelength/slit width of 435/20 nm or 485/20 nm, and emission slit width of 5 nm. In other cases, proteolysis was quantified in 96/384-well black plates using either a Biotek synergy4 or FLx800 reader (Winooski, Vt.). These instruments were equipped with filters for Cerulean (Ex420/50 nm and Em485/20 nm) and Venus (Ex485/20 nm and Em540/25 nm). FRETS-VWF73 was read on the FLx800 reader equipped with Ex360/40 and Em 460/40 filters. A parameter termed “FRET ratio” was used to quantify the extent of VWF-A2 FRET proteolysis. This is defined as the ratio of emitted light intensity at 485 nm (primarily by Cerulean) versus 540 nm (primarily by Venus), when the protein was excited at 420 nm. This parameter is inversely related to FRET efficiency and it increases upon VWF-A2 proteolysis.

Western Blot Analysis:

SDS-PAGE under standard reducing conditions was performed using either 4-20% gradient gels (Thermo-Fisher, Rockford, Ill.) for VWF-A2 FRET or 6% resolving gels for plasma VWF. Proteins were then transferred onto nitrocellulose membranes. These were detected using either goat anti-his polyclonal Ab (Bethyl laboratories, Montgomery, Tex.) for VWF-A2 FRET, or rabbit anti-human VWF polyclonal Ab (Dako, Carpinteria, Calif.) for plasma VWF. Silver staining was performed using a kit from Thermo-Fisher.

Statistics:

Unless stated otherwise, data are presented as mean±standard error of mean for ≧3 replicate experiments. ANOVA was applied for comparison between multiple treatments. * p<0.05 was considered significant.

### Example 2

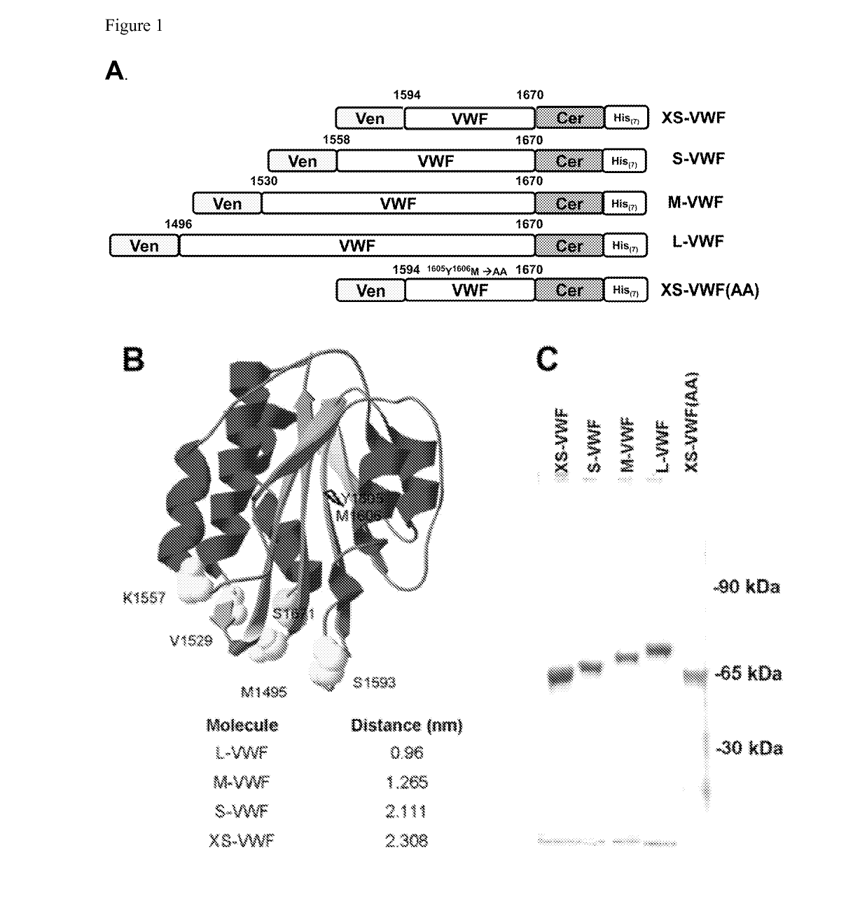

This Example provides a demonstration that truncated VWF-A2 constructs exhibit FRET properties. In order to show this, the entire VWF-A2 domain or fragments of this domain were engineered to express Venus at the N-terminus, and Cerulean at the C-terminus (FIG. 1A). All proteins with Venus and Cerulean were named based on the size of the inserted VWF-A2 fragment. Venus insertion sites were located in loop regions between β-sheets/α-helices based on X-ray crystal data (Q. Zhang, et al. Proc Natl Acad Sci USA 106 (2009) 9226-31) (FIG. 1B). Although the GFP variant insertion sites were located 77-175 amino acids from each other, in all proteins, these sites are located within 2.5 nm in the crystal. Thus, the proteins were engineered to exhibit FRET properties. In addition, in the control protein, Y¹⁶⁰⁵ and M¹⁶⁰⁶ in the cleavage sequence in the smallest FRET construct (XS-VWF) were both mutated to alanine. All proteins were enriched using Ni-NTA columns to >95% purity as assessed using silver staining of SDS-PAGE gels (FIG. 1C).

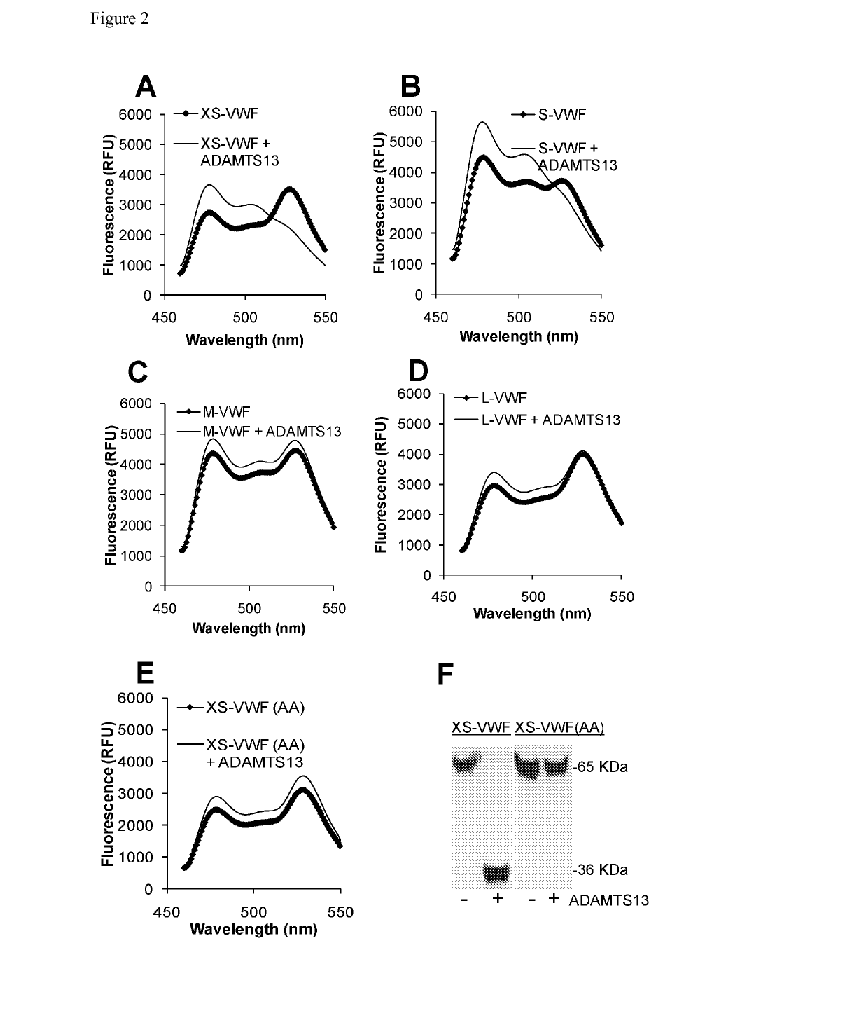

All proteins exhibited FRET properties (FIG. 2). FRET ratio varied from 0.74 for L-VWF to 1.24 for S-VWF. This corresponds to FRET efficiencies of 31% and 19% respectively. Such variation is consistent with the notion that in addition to the spatial distance separating the GFP-variant insertion sites, the distance between these fluorophores in the fusion protein and their relative orientation also determine the energy transfer efficiency. Upon addition of ADAMTS13, FRET ratio increased from 0.78 to 1.73 for XS-VWF (FIG. 2A) and from 1.24 to 1.84 for S-VWF (FIG. 2B) within 1 h. Complete cleavage corresponds to a FRET ratio of ˜2.2. There was very little change in the FRET ratio of M-VWF (FIG. 2C), L-VWF (FIG. 2D), and XS-VWF(AA) (FIG. 2E) upon addition of ADAMTS13. Western blot analysis of XS-VWF and XS-VWF(AA) (FIG. 2F) is consistent with the fluorescence measurements, and it confirms that changes in FRET signal of XS-VWF is primarily due to the cleavage of the Y¹⁶⁰⁵-M¹⁶⁰⁶ scissile bond by ADAMTS13.

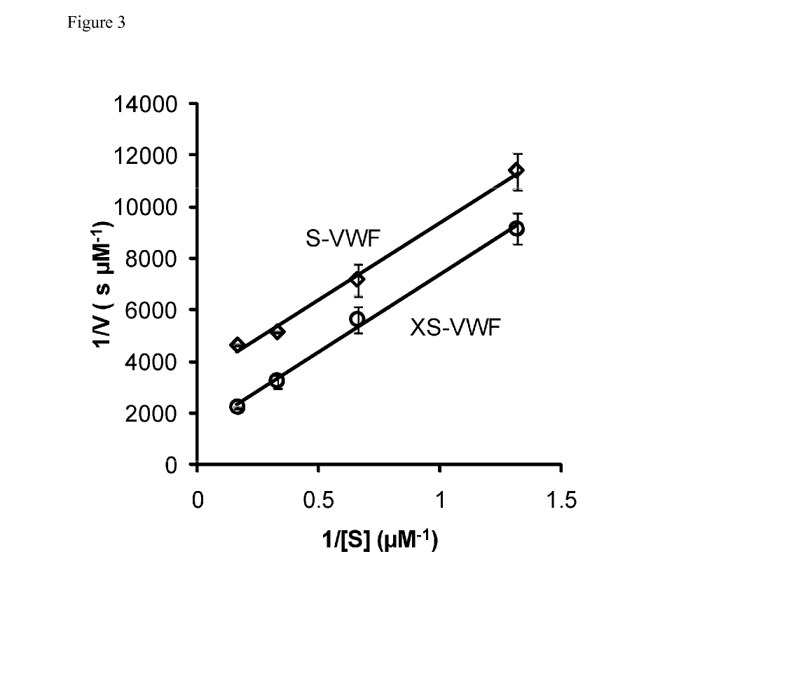

The K_{M} and k_{cat} values of XS-VWF and S-VWF were quantified based on the Lineweaver-Burk plot in the presence of 20% human plasma (FIG. 3). These results show that both parameters were lower for S-VWF in comparison to XS-VWF (Table 3). Further, the measured K_{M} and k_{cat} values were similar to previously published values for other proteins constructed based on the VWF-A2 domain.

TABLE 3

| K_{M} | | k_{cat} (min⁻¹) |
|---|---|---|
| 4.6 ± 0.8 | μM | 44.8 ± 4.3 |
| 1.8 ± 0.2 | μM | 17.7 ± 0.5 |
| 3.2 ± 1.1 | μM | 58 |
| 1.6 ± 0.5 | μM | 8.4 ± 3.6 |
| 15 | nM | 0.83 |

### Example 3

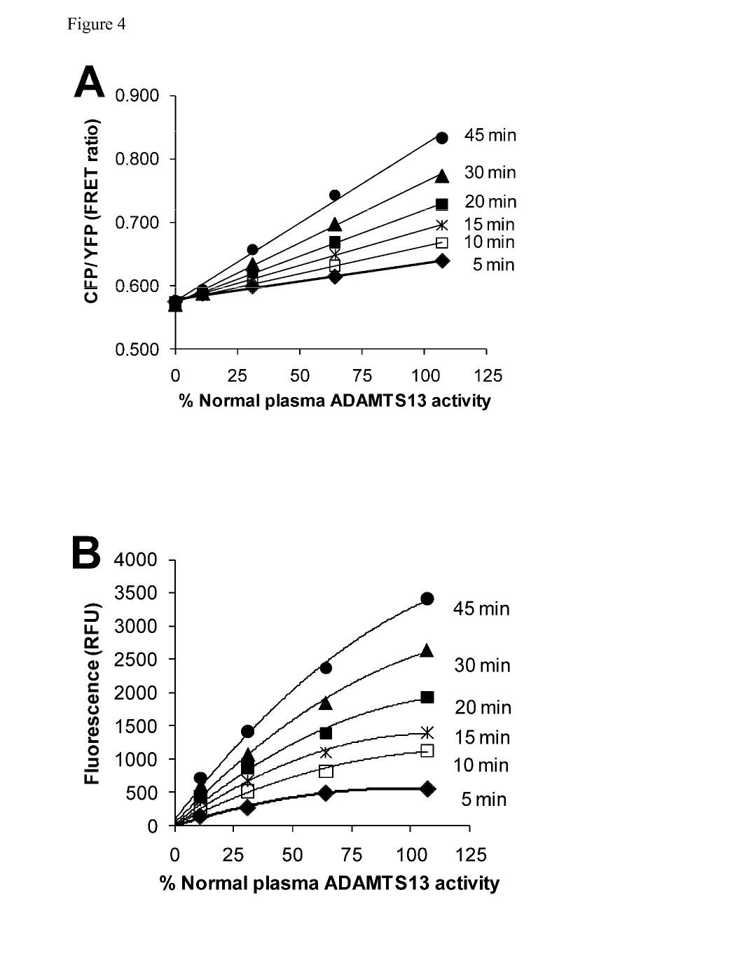

This Example provides a comparison of XS-VWF with FRETS-VWF73. To perform this comparison we tested the ability of one of the VWF-A2 FRET proteins, XS-VWF, to detect ADAMTS13 activity in human plasma samples. Upon comparing cleavage rates of XS-VWF with FRETS-VWF73 using samples having a range of plasma ADAMTS13 activity and measured over time, we noted a linear change in the FRET ratio of XS-VWF with ADAMTS13 activity (FIG. 4A). Under identical conditions, FRETS-VWF73 fluorescence readout exhibited a non-linear relationship with plasma ADAMTS13 activity (FIG. 4B).

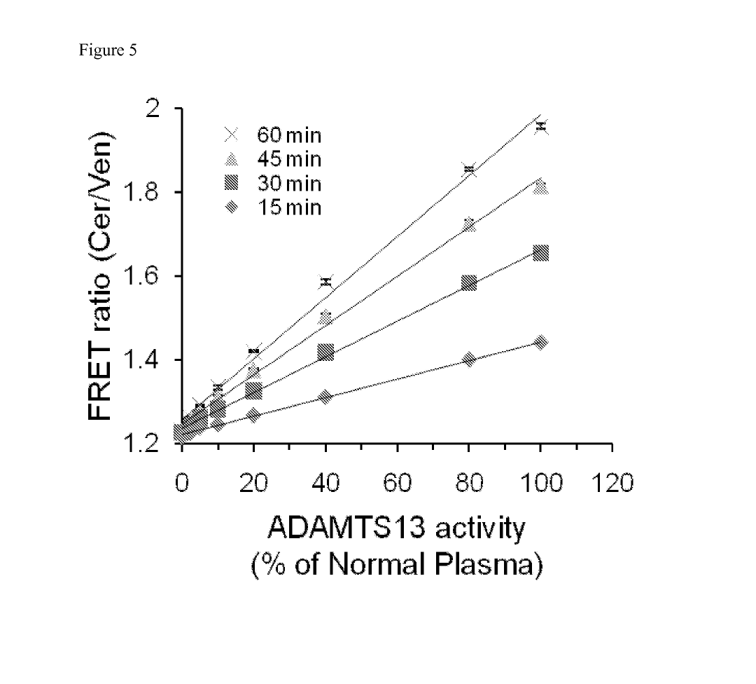

When using normal human plasma, XS-VWF could be used to detect ADAMTS13 activity within 5 min, the earliest measured time point. Thus, in one embodiment, the invention provides for first detecting ADAMTS13 activity in normal plasma in not more than 5 minutes. In the presence of lower ADAMTS13 activity, more time was required for reliable detection of enzyme activity. Since auto-fluorescence due to plasma proteins plays only a minor role in assays utilizing XS-VWF, higher plasma concentrations can be used for this substrate compared to FRETS-VWF73 which exhibits higher auto-fluorescence. When assays were performed by diluting 204 of a plasma mixture containing various ratios/amounts of normal plasma (100% ADAMTS13 activity) and heat-inactivated plasma (0% ADAMTS13 activity) in 100 μL reaction volume, 10% of normal plasma protease activity could be detected within 30 min (FIG. 5).

In order to further test our reagent, we compared substrate proteolysis with a panel of human plasma samples that were collected in sodium citrate (Table 4).

TABLE 4
XS-VWF versus FRETS-VWF73

% normal plasma
ADAMTS13 activity

| | | FRETS- |
|---|---|---|
| Sample | XS-VWF | VWF73 |
| CalA [AV = 0%] | −0.1 ± 0.14 | −3.0 ± 0.7 |
| CalB [AV = 11%] | 8.3 ± 1 | 15.2 ± 1.4 |
| CalC [AV = 31%] | 32.9 ± 0.3 | 31.6 ± 0.7 |
| CalD [AV = 64%] | 66.7 ± 0.2 | 60.9 ± 0 |
| CalE [AV = 107%] | 104.8 ± 2.5 | 108.9 ± 0.3 |
| Plasma 1 | 5.8 ± 0.2 | 22.9 ± 0.6 |
| Plasma 1 (HI) | 0.6 ± 1 | 18.6 ± 0.3 |
| Plasma 1 mixed | 46.7 ± 0.1 | 46.9 ± 0.1 |
| Plasma 2 | −3.2 ± 1.1 | 14.2 ± 1 |
| Plasma 2 (HI) | −1.4 ± 1.2 | 13.4 ± 0.8 |
| Plasma 2 mixed | −1.6 ± 1.2 | 16.2 ± 2.1 |
| Plasma 3 | 103.2 ± 2.2 | 105.2 ± 3 |
| Plasma 3 (HI) | −3.0 ± 1.4 | 9.6 ± 7.8 |
| Plasma 3 mixed | 48.9 ± 0.4 | 46.5 ± 1.1 |
| Plasma 4 | 7.3 ± 1.8 | 12.4 ± 0.2 |
| Plasma 5 | 25.8 ± 6.3 | −1.6 ± 1.6 |
| Plasma 6 | 21.0 ± 5.4 | −4.6 ± 3.4 |
| Plasma 7 | 24.9 ± 0.6 | 36.9 ± 1.6 |
| Plasma 8 | 70.4 ± 14.9 | 32.1 ± 1.41 |
| Plasma 9 | 67.4 ± 14.35 | 32.9 ± 1.3 |
| Plasma 10 | 114.3 ± 23.4 | 80.7 ± 3.8 |
| Plasma 11 | 116.0 ± 23.7 | 88.7 ± 2.1 |
| Plasma 12 | 93.7 ± 0.4 | 80.9 ± 1.2 |

* ADAMTS13 activity was measured using either XS-VWF or FRETS-VWF73 as substrate for up to 45 min. Measured fluorescence value was converted to % ADAMTS13 activity using calibration curve in FIG. 4. Data from 30 and 45 min were averaged, and are presented as Mean ± standard deviation.

The test samples include: i) Five calibration standards that had ADAMTS13 activity values (AVs) assigned based on the dilutions of a pool of normal plasma collected by the ISTH [International Society on Thrombosis and Haemostasis]; ii) Proficiency specimens obtained from individuals deficient in ADAMTS13 activity (Plasma 1), individuals with anti-ADAMTS13 antibodies (Plasma 2), and normal control (Plasma 3). For these three samples, in some cases, ADAMTS13 activity was heat inactivated by incubating plasma at 56° C. for 30 min (specimens identified as “HI” in Table 4). In other runs (identified as ‘mixed’), heat inactivated plasma was mixed with an equal volume of normal plasma in order to check for anti-ADAMTS13 inhibitory antibodies iii) Six Factor Assay ConTrol plasma from George King Biomedical (Overland Park, Kans.): two FACT (plasma 11, 12), two A-FACT (plasma 5, 6) and two B-FACT (plasma 8, 9) samples that exhibit normal, abnormal and borderline activity in a wide variety of coagulation tests. iv) Additional human plasma specimens exhibiting a range of ADAMTS13 activity levels (plasma 4, 7, 10). As can be seen (Table 4), the results from XS-VWF activity measurements were largely consistent with findings noted using FRETS-VWF73. XS-VWF consistently gave lower values in the case of patients with <20% ADAMTS13 levels while FRETS-VWF73 appears to over predict this (Table 5 and also FIG. 5). This is particularly noted in case of heat inactivated plasma, where XS-VWF reported values close to zero while FRETS-VWF73 reported values ˜14%. Thus, when plasma ADAMTS-13 concentration is low, XS-VWF is a more sensitive substrate compared to FRETS-VWF73. Differences were also noted in some plasma samples, namely 5,6 and 8,9. The reason for this has not been currently identified though it may be attributed to inherent structural differences in the two substrates and the manner in which ADAMTS13 binds them.

### Example 4

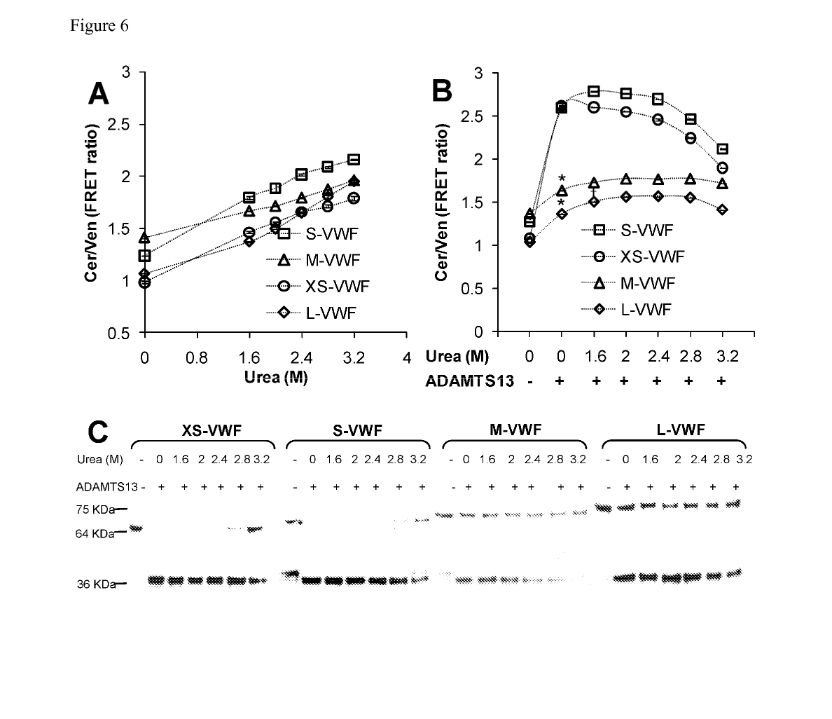

This Example provides a demonstration of the effects of urea on VWF-A2 folding and access to the proteolysis site. Since ADAMTS13 alone could not cleave M- and L-VWF (FIG. 2), studies were performed where urea concentration in the cleavage buffer was varied from 0 to 3.2M. Experiments were performed both in the absence (FIG. 6A) and presence (FIG. 6B-C) of ADAMTS13. In FIG. 6A, FRET ratio increased approximately linearly with urea concentration even in the absence of ADAMTS13. Over this range of urea concentration, there was negligible change in the fluorescence properties of Venus or Cerulean alone. Significant structural changes occur at urea concentration down to 1.6M.

ADAMTS13 was added to the VWF-A2 FRET proteins in the presence of varying urea concentrations. FRET ratio measurement (FIG. 6B) and western blot analysis (FIG. 6C) of selected samples was performed. Complete cleavage of XS-VWF and S-VWF was observed at urea concentrations <2.8M. The data confirm that urea is not required for the cleavage of these proteins and that ADAMTS13 activity is diminished at high urea concentrations. In the absence of urea, the extent of cleavage of both L- and M-VWF was 20%. Increasing urea concentration to 1.6M resulted in 30% proteolysis. Complete proteolysis was not observed under any condition. Western blot analysis results (FIG. 6C) are consistent with the fluorescence measurements.

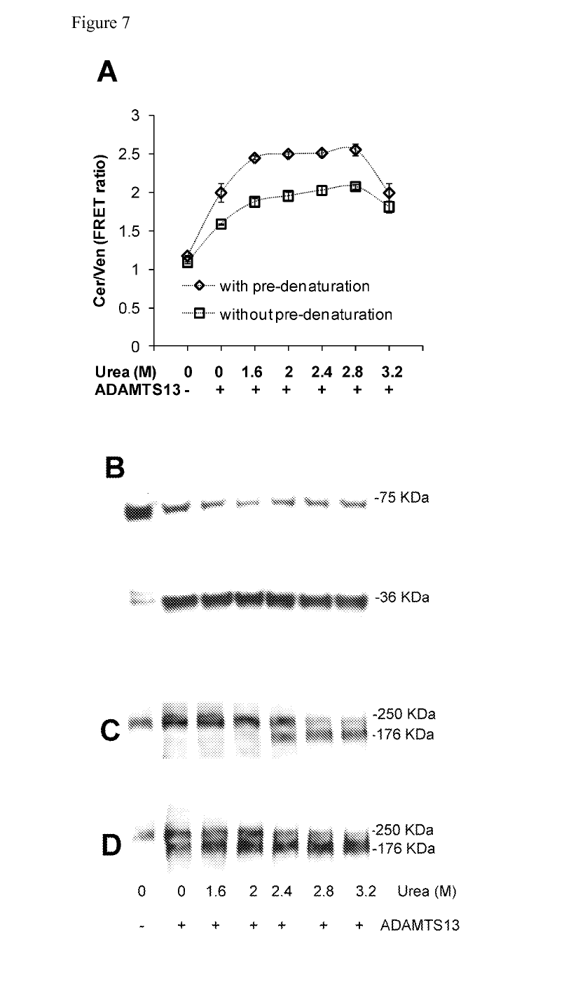

Since complete cleavage of L-VWF was not observed under any condition, we further increased the urea concentration by incubating L-VWF and multimeric human plasma VWF with 4M urea for 1 h prior to dilution into a solution which contained ADAMTS13 along with lower urea concentrations (FIG. 7). Here, we noted substantial cleavage of L-VWF even when the protein was diluted into buffer that lacked urea. >90% cleavage of L-VWF was observed at 2M urea upon inclusion of the pre-denaturation step (FIG. 7A, 7B). Similar to L-VWF, multimeric VWF was also not fully cleaved when the protein was incubated with varying urea concentrations and ADAMTS13 (FIG. 7C). Proteolysis was however complete at urea>2.4M, provided a pre-denaturation step was included (FIG. 7D). The observation suggests that the proteolysis of L-VWF proceeds under similar denaturation conditions as the plasma protein.

### Example 5

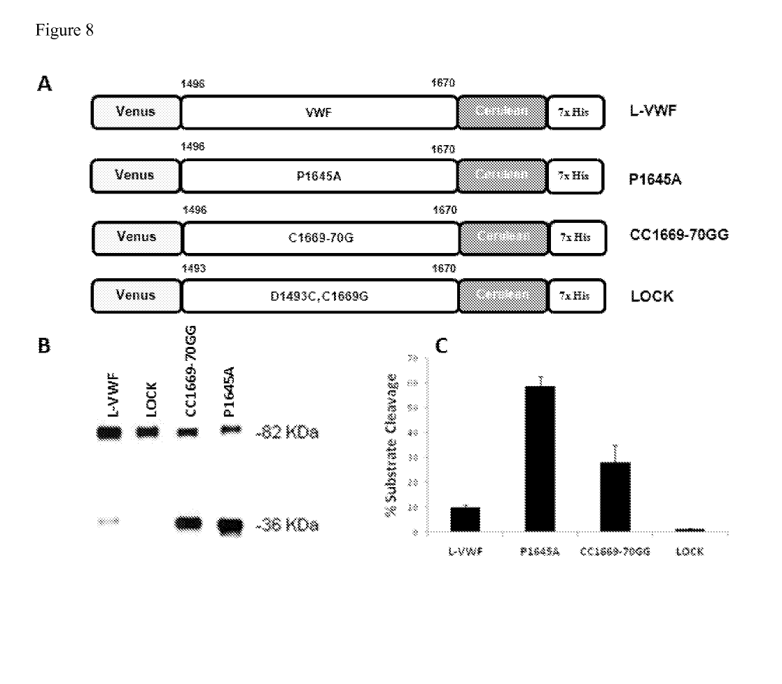

This Example provide a description of progressive denaturation of VWF-A2 with varying urea concentrations and also examples amino acid substitutions that are designed to illustrate the selection of mutations outlined previously in Table 1. In this regard, while the smallest FRET protein is well suited for measuring ADAMTS13 activity, the largest molecule (L-VWF) provides insight into the dynamics of VWF conformation change upon addition of denaturant. In this regard, others have used urea as a surrogate for fluid shear since this denaturant exposes the cryptic Tyr¹⁶⁰⁵-Met¹⁶⁰⁶ cleavage site in VWF-A2 (M. Auton, et al. J Mol Biol 366 (2007) 986-1000). The present data show that protein conformation changes progressively with urea concentration which is consistent with the notion that multiple salt bridges stabilize the A2 domain. Alternatively, VWF-A2 FRET may exist in only a limited number of states with urea regulating the distribution of the protein in these states. In connection with the VWF-A2 FRET protein variants described herein, FIG. 8A provides a schematic of specific VWF-A2 FRET proteins that contain single amino acid substitutions. We tested the cleavage of these substrates using recombinant ADAMTS13 in the presence of 1M urea (Western blot results presented in FIG. 9B). It is notable that changes in the Pro1645 site enhances VWF cleavage even in the presence of low concentrations of urea that are insufficient to cleave the wild-type protein. Similarly, substitution of Cysteine by Glycine at positions 1669 and 1670 enhances VWF A2 domain proteolysis. The principles laid out in FIG. 9B are in agreement with FRET data presented in FIG. 9C which demonstrate higher cleavage of mutant VWF proteins compared to the wild-type molecule upon addition of low concentrations of urea.

It will be apparent from the foregoing description, the Examples and the Figures that the polypeptides of the invention, and XS-VWF in particular, can be used as rapid, reliable, sensitive and cost-effective reagents to monitor plasma ADAMTS13 activity and can detect ADAMTS13 activity below 10% normal levels within 30 min. This type of reagent is important since it provides a measure of the hemostatic potential of blood. In addition to TTP, decreased ADAMTS13 activity is also associated with poor prognosis during sepsis-induced organ failure and with increased risk of non-fatal heart attack. Reagents that detect ADAMTS13 activity can also be applied to distinguish TTP from other unrelated disorders like hemolytic uremic syndrome (HUS), which present similar clinical symptoms.

While the invention has been described through illustrative examples, routine modifications will be apparent to those skilled in the art, which modifications are intended to be within the scope of the invention.

We claim:

1. A recombinant polypeptide comprising sequentially i) a first fluorescent protein ii) a human von Willebrand factor (VWF) amino acid sequence which comprises at least amino acids 1596-1668, inclusive, of SEQ ID NO:1 and, iii) a second fluorescent protein.

2. The recombinant polypeptide of claim 1, wherein the VWF amino acid sequence is not longer than 229 amino acids.

3. The recombinant polypeptide of claim 2 comprising at least one mutation selected from the group of mutations in Table 1 wherein the mutation occurs at a VWF amino acid position between VWF amino acid positions 1596-1668.

4. The recombinant polypeptide of claim 3, wherein at least one mutation is a change in Pro 1645 to any amino acid that is not Pro.

5. The recombinant polypeptide of claim 2 comprising at least one mutation selected from the group of mutations in Table 1 wherein the mutation occurs at a VWF amino acid position between VWF amino acid positions 1594-1670.

6. The recombinant polypeptide of claim 5, wherein the at least one mutation is a change in Pro 1645 to any amino acid that is not Pro.

7. The recombinant polypeptide of claim 2 comprising at least one mutation selected from the group of mutations in Table 1 wherein the mutation occurs at a VWF amino acid position between VWF amino acid positions 1458 to 1686.

8. The recombinant polypeptide of claim 7, wherein the at least one mutation is a change in Pro 1645 to any amino acid that is not Pro.

9. A composition comprising the recombinant polypeptide of claim 1 and an ADAMTS13 protease.

10. The composition of claim 9 further comprising a denaturant, wherein the denaturant is in a concentration of not more than 4M.

11. The composition of claim 10, further comprising blood, bodily fluids, or isolated plasma.

12. A method for determining ADAMTS13 protease activity comprising contacting a recombinant polypeptide according to claim 1 with a composition comprising ADAMTS13 protease and analyzing ADAMTS13 cleavage of the recombinant polypeptide using Fluorescence Resonance Energy Transfer (FRET) analysis of fluorescence from the first and second fluorescent proteins, wherein the cleavage occurs between Tyr 1605 and Met 1606 Met of the recombinant polypeptide, and wherein a change in ratio of the fluorescence from the first and the second fluorescent proteins over time is indicative of ADAMTS13 protease activity.

13. The method of claim 12, wherein the von Willebrand factor (VWF) amino acid sequence comprises the sequence of SEQ ID NO:2.

14. The method of claim 13, wherein the VWF amino acid sequence is not longer than 229 amino acids.

15. The method of claim 14, wherein the VWF amino acid sequence comprises at least one mutation selected from the group of mutations in Table 1 wherein the mutation occurs at a VWF amino acid position between VWF amino acid positions 1458 and 1686.

16. The method of claim 15, wherein the at least one mutation is a change in Pro 1645 to any amino acid that is not Pro.

17. The method of claim 12 wherein the composition comprising the ADAMTS13 protease comprises a biological sample.

18. The method of claim 17, wherein a lower FRET ratio from the first and the second fluorescent proteins relative to a reference FRET ratio is indicative that the individual from whom the biological sample was obtained has or is at risk for a disease that is positively correlated with abnormally high molecular weight VWF in the blood of the individual.

19. The method of claim 18, wherein the disease is thrombotic thrombocytopenic purpura (TTP).


SEQUENCE LISTING (19 sequences)

SEQ ID NO: 1 - protein, 2813 aa, human
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 2 - protein, 73 aa, human
DREQAPNLVYMVTGNPASDEIKRLPGDIQVVPIGVGPNANVQELERIGWPNAPILIQDFETLPREAPDLVLQR

SEQ ID NO: 3 - protein, 77 aa, human
QGDREQAPNLVYMVTGNPASDEIKRLPGDIQVVPIGVGPNANVQELERIGWPNAPILIQDFETLPREAPDLVLQRCC

SEQ ID NO: 4 - protein, 229 aa, human
CDLAPEAPPPTLPPHMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDC

SEQ ID NO: 5 - DNA, 36 bp, artificial sequence
  PCR primer
gggcggaccggtatggtgagcaagggcgaggagctg

SEQ ID NO: 6 - DNA, 36 bp, artificial sequence
  PCR primer
cgcgccaccggtcttgtacagctcgtccatgccgag

SEQ ID NO: 7 - DNA, 38 bp, artificial sequence
  PCR primer
cgcgcgcaagcttaatggtgatggtgatggtgatggcc

SEQ ID NO: 8 - DNA, 34 bp, artificial sequence
  PCR primer
gcggcggcatatggtgagcaagggcgaggagctg

SEQ ID NO: 9 - DNA, 35 bp, artificial sequence
  PCR primer
cgcgcaccggtctcgtccatgccgagagtgatccc

SEQ ID NO: 10 - DNA, 27 bp, artificial sequence
  PCR primer
aacatggtgagcaagggcgaggagctg

SEQ ID NO: 11 - DNA, 34 bp, artificial sequence
  PCR primer
ccgccgttcgaacttgtacagctcgtccatgccg

SEQ ID NO: 12 - DNA, 35 bp, artificial sequence
  PCR primer
gtgtgtaccggtcagggtgatcgggagcaggcgcc

SEQ ID NO: 13 - DNA, 34 bp, artificial sequence
  PCR primer
cgtagtaccggtaaaggggacatcctgcagcggg

SEQ ID NO: 14 - DNA, 34 bp, artificial sequence
  PCR primer
cgcgcgaccggtgtgggccaggacagcatccacg

SEQ ID NO: 15 - DNA, 33 bp, artificial sequence
  PCR primer
gggattaccggtgttctggatgtggcgttcgtc

SEQ ID NO: 16 - DNA, 25 bp, artificial sequence
  PCR primer
aacgcagcacctctgcagcaccagg

SEQ ID NO: 17 - DNA, 37 bp, artificial sequence
  PCR primer
gcgcgcgcatatggggctcttgggggtttcgaccctg

SEQ ID NO: 18 - DNA, 27 bp, artificial sequence
  PCR primer
aaccctctgcagcaccaggtcaggagc

SEQ ID NO: 19 - DNA, 29 bp, artificial sequence
  PCR primer
gatttcccgtgacagcagcgaccaggttg